(19) United States
(12) Patent Application Publication
(10) Pub. No.: US 2011/0070258 A1
(43) Pub. Date: Mar. 24, 2011

(54) METHOD OF SLOWING THE AGING PROCESS BY ACTIVATING SIRTUIN ENZYMES

(75) Inventors: Miguel JIMENEZ DEL RIO, Las Palmas (ES); Paul Flowerman, Morristown, NJ (US); Vladimir Badmaev, Staten Island, NY (US)

(73) Assignee: Polifenoles Naturales, S.L., Ingenio (ES)

(21) Appl. No.: 12/885,271

(22) Filed: Sep. 17, 2010

Related U.S. Application Data
(60) Provisional application No. 61/243,828, filed on Sep. 18, 2009.

(51) Int. Cl. A61K 36/03; A61K 31/336; A61K 36/185; A61P 39/00; A61P 3/00

(52) U.S. Cl. 424/195.17; 514/475; 424/725

(57) ABSTRACT

The fucoxanthin/pomegranate seed oil composition describes a method of slowing the aging process in a mammalian subject by activating at least one member of the sirtuin family of proteins, wherein the activating step includes administering to the subject a synergistic combination of fucoxanthin and punicic acid. Sirtuin enzymes exert their function by removing acetyl groups from proteins. The deacetylation results in inactivation of the proteins' role in cell metabolism and prevents genes from over-expression, thereby putting a cell into a state of hibernation and increasing its lifespan.

### RELATED APPLICATION

This application claims the benefit of U.S. Provisional Application No. 61/243,828, filed on Sep. 18, 2009, the entire disclosure of which is incorporated herein by reference.

### FIELD

This disclosure relates to a composition for slowing the aging process in a mammalian subject. This disclosure further relates to a composition for activating sirtuin enzymes.

### BACKGROUND

Sirt1 is an enzyme which deacetylates proteins that contribute to cellular regulation. The enzyme sirtuin-1 (Sirt1) is involved in the molecular mechanisms linking lifespan to adipose tissue. Sirt1 plays a key modulatory role in animal fat deposition, is involved in adipogenesis, and promotes fat mobilization in white adipocytes. Sirtuin-1 regulates several transcription factors that govern fat metabolism, including peroxisome proliferator-activator receptor-γ(PPAR-γ), fork-head-box transcription factors, and adiponectin. The activation of Sirt1 promotes fat mobilization by repressing PPAR-γ, one of the transcriptional factors in fat storage. Sirt1 inhibits lipid accumulation in adipocytes.

The sirtuin gene is significant in the suppression of DNA instability and it also allows the DNA to be repaired. Aging is in part caused by the inability of older cells to replicate DNA perfectly in every new cell, like it could when we are younger. This results in DNA debris, which causes aging by accelerating death of individual cells. Evidence links Sirt1 with aging.

The predominant type of fat in the body is white adipose tissue (WAT), which functions to store energy in the form of triglyceride (TG) intracellular droplets and to secrete several cytokines (adipokines) that regulate overall energy balance by affecting the function of other tissues and organs such as the brain, muscle, and liver. Adipokines include leptin, adiponectin, visfatin, resistin, interleukin (IL)-6 and tumor necrosis factor-alpha (TNF alpha) which regulate energy metabolism, insulin sensitivity, and cardiovascular health. When energy is required, the stored TG is hydrolyzed via activation of lipolytic pathways. The coordination of TG storage and utilization is regulated by the family of protein compounds, perilipins, which coat TG droplets and allow or prevent access of the lipolytic enzymes. The other type of fat is brown adipose tissue (BAT) whose principal function is to burn TG released (lipolysis) fatty acids to generate heat energy (“non-shivering” thermogenesis), particularly in newborns as a protective measure during the initial hours following birth into a cold environment. BAT depots decrease significantly in size as humans mature, existing in adults within small pockets throughout the body as well as distributed within WAT deposits.

Brown adipose tissue occurs in humans up until the 8th decade of life. The functional distinction between white and brown fat tissues is that only brown fat is equipped for the rapid oxidation of the products of lipolysis from the fat reserves. WAT consists of unilocular cells filled with a single fat droplet, whereas BAT consists of multilocular lipid storage, caused by the rapid oxidation of fat. The active BAT is also heavily innervated. The experimental denervation of BAT results in many unilocular cells resembling white fat cells. BAT cells are smaller than those of white adipose tissue and BAT is found in characteristic locations. BAT can be found in humans close to the neck vessels and muscles, under the clavicles and axillae, around intercostal vessels, between the trachea and the esophagus, in the para-aortic region as well as in the perirenal and suprarenal regions. The brown fat in the interscapular area disappears, gradually, up to 30 years of age, and sharply thereafter.

BAT is present in relatively large amounts (2-5%) of body weight in a newborn and it is generally considered to atrophy with aging, possibly being converted into white adipose tissue. The decline in BAT parallels decline in the capacity for non-shivering thermogenesis and predisposition to accumulate WAT (e.g. central obesity) and increase in total body weight. The studies of adipose tissue suggest that BAT with aging is less active based on morphological appearance that is different from that of BAT in a newborn or of cold-adapted rodents. There is also evidence that BAT may be more pronounced and active in men depending on adverse atmospheric conditions, such as exposure to cold. For example, BAT has been found more extensively distributed in the bodies of Finnish men who had lived outdoors.

Diet and age have important influences on BAT. Caloric restriction (CR) has been shown to prevent the age-associated loss of mitochondrial function and biogenesis in several tissues such as liver, heart, and skeletal muscle and prevents the age-associated decline in mitochondrial function in BAT, probably in relation with less impairment of mitochondrial biogenesis. BAT mitochondria obtained from 24-month-old male and female rats previously subjected to 40% CR diet for 12 months were compared with mitochondria from old (24 months) and young (6 months) ad libitum fed rats. Old restricted rats compared to old ad libitum fed ones showed a reduction in BAT size with respect to fat content and adipocyte number.

Brown adipocytes in young individuals contain large numbers of mitochondria and are densely innervated by the sympathetic nervous system. These nerve endings release noradrenaline into the surroundings of fat cells, where noradrenaline activates G-protein-coupled beta-adrenergic receptors which triggers metabolic events activating of uncoupling protein 1 (UCP1). Activation of Sirt1 induces expression of UCP1. The UCP1 is a signature molecule of BAT. Uncoupling protein 1 is a unique feature of brown adipocytes that allows for the generating of energy upon activation of parasympathetic and sympathetic nervous systems by environmental factors, e.g. cold, food and endogenous stimulation via metabolic hormones, e.g. glucocorticoids. UCP1 is found in the inner membrane of the mitochondrion, where uncoupling protein 1 uncouples the oxidation of fuel from adenosine triphosphate (ATP) production which leads to non-shivering thermogenesis. Increased levels of UCP1 may be seen as synonymous with active BAT.

The metabolic functions of the body, including BAT and WAT, depend to a large degree on the cellular “powerhouses,” the mitochondria. The viable mitochondria are maintained by the sirtuin family of proteins, the class of proteins highly conserved evolutionarily with the single function of safeguarding cell survival. The sirtuins of simple organisms, e.g. Sirt2 in yeast, and the sirtuins of mammals, i.e., mammalian Sirt1-7 proteins, are up-regulated under calorie-restricted (CR) diet, adverse environmental conditions, e.g. harsh and cold weather, and hibernation. Interestingly, CR diet is the only proven way of extending the life span of an organism, from bacteria to humans. Studies in model organisms, e.g. yeast, Drosophila fly, nematodes, or rodents suggest that two evolutionary pathways may increase longevity: (a) repair of life encoding genetic material, and (b) economizing the metabolic activity to minimize the collateral damage and wear and tear effects of life sustaining metabolism.

Sirtuin enzymes, e.g. Sirt1 and Sirt3, exert their function by removing the acetyl group from proteins. The deacetylation results in inactivation of the proteins' role in cell metabolism and prevents genes from over-expression, thereby putting a cell into a state of hibernation (less wear and tear and more time for the repair), hence potentially increasing its lifespan. The cell uses the acetylation/deacetylation in similar way as phosphorylation/dephosphorylation, to activate or deactivate proteins. The mechanism of deacetylation is regulated by interaction among Sirt1-7 proteins whose functions are nicotine amide (NAD) dependent. In general, sirtuin activation is accompanied by an increase in the levels of NAD. The NAD dependent mechanism of deacetylation regulates metabolism and support cell longevity. The ratio between NAD and its reduced form NADH is related to caloric intake. The increased levels of NADH mean more caloric intake, because the energy in the food is translated to the NADH, which then is used to generate energy in form of ATP. On the other hand, the increase in the NAD/NADH ratio occurs in the CR diet.

Activation of Sirt1 by administration of a calorie-restricted diet or resveratrol has been extensively studied, and Sirt1 has been linked to aging. Researchers have compared mice that were fed diets with various restrictions and either a high or low dose of resveratrol. Higher doses of resveratrol directly increased levels of sirtuin or Sirt1. Resveratrol was found to prevent obesity and age related cardiovascular decline in mice. It also had several positive effects on age-related conditions such as an improved balance and motor coordination, fewer cataracts, better bone thickness, density, and mineral content. The ageing process was slowed, age related diseases declined, and life was actually prolonged. Lower doses of resveratrol have since been found to show the same effect on the sirtuin gene. Activation of Sirtuins suppresses peroxisome proliferator-activated receptor gamma, PPAR-γ, and attenuates adipogenesis as well as triggering lipolysis and loss of WAT. Conversely inhibition of Sirtuins increases WAT formation.

The reduction in WAT extends murine lifespan, and this finding suggests a possible molecular pathway connecting caloric restriction to life extension in mammals. The mitochondrial localization of Sirt3-5 is especially important because mitochondrial dysfunction is associated with mammalian aging and metabolic diseases, including diabetes, neurodegenerative diseases, and cancer. The Sirt3 is expressed in brown adipose tissue and the deacetylase activity of Sirt3 is reported to be required for the induction of BAT and its signature uncoupling protein 1 (UCP-1). Sirt3 regulates mitochondrial functions, and its over-expression increases respiration, while decreasing reactive oxygen species production. In human population studies, polymorphisms within the Sirt3 gene (a mechanism to safeguard the valuable gene) have been linked to longevity.

The ectopic expression of BAT uncoupling protein 1 in mouse skeletal muscle and induction of UCP1 in mouse or human white adipocytes promote fatty acid oxidation and resistance to obesity. The importance of brown adipose tissue in the regulation of energy balance in man cannot be underestimated. On one hand BAT has been found to prevent obesity; on the other hand, its anti-obesity mechanism uniquely operates through and is interlinked with enhancement of Sirt1-7 proteins which increase longevity. It has been found that a 20-25% increase in metabolic rate could be accomplished by as little as 40 to 50 gm of active BAT. This amount of BAT translates to less than 0.1% of the average human body weight. The 20% difference in daily energy expenditure could make a difference between maintaining body weight or gaining at the rate of 20 kg per year. This finding is especially relevant in the aging of the human organism, since an average adult gains 0.45 kg (1 lb) per year after age 25 and loses 0.2 kg (0.5 lb) of muscle and bone mass each year after age 25.

Plant phenolics, including phytoalexins, i.e., resveratrol present in grape skin, can stimulate sirtuin enzymes, which in turn may slow down the aging process and may also regulate the metabolic process. In the in vitro experiments with adipocytes, resveratrol may affect the expression of several adipogenic transcription factors and enzymes, e.g. downregulating the peroxisome proliferator-activated receptor PPART gamma, C/EBP alpha, SREBP-1c, FAS, HSL, LPL genes and up-regulating expression of genes responsible for mitochondrial activity, i.e. Sirt3, UCP1 and Mfn2 (Mitofusin 2).

These in vitro findings indicate that resveratrol may alter fat mass by directly affecting cell viability and adipogenesis in maturing pre-adipocytes and inducing apoptosis in adipocytes, and thus may have applications for the treatment of obesity. However, the potential role of natural compounds like resveratrol and other plant phenolics in regulation of metabolism and as anti-obesity compounds is diminished by their poor gastrointestinal absorption, tissue bioavailability evidenced predominantly in vitro and animal studies. In addition, resveratrol may contribute to increased blood levels of a cardiovascular disease risk factor, homocysteine, and some phenolic compounds, e.g. tea polyphenols in large quantities maybe related to hepatotoxicity.

### SUMMARY

The prevalence of obese individuals in the US almost tripled between years 1960 and 2000, and available pharmacological and nutritional interventions have failed to slow this trend. Various exemplary embodiments of the fucoxanthin/pomegranate seed oil, composed of standardized plant extracts, provides a safe and effective method to increase resilience to obesity and its metabolic consequences by:
preventing age related loss of BAT,
increasing volume of brown adipose tissue at the expense of white adipose tissue, and
slowing down the aging process.

Various exemplary embodiments relate to use of a synergistic mixture in the manufacture of a medicament for slowing the aging process in a mammalian subject by activating at least one member of the sirtuin family of proteins. The synergistic mixture comprises:
(a) an effective amount of fucoxanthin, a pharmaceutically acceptable salt of fucoxanthin, or a mixture thereof; and
(b) an effective amount of punicic acid.

Various exemplary embodiments relate to use of a synergistic mixture of fucoxanthin and punicic acid in the manufacture of a medicament for activating at least one member of the sirtuin family of proteins. Various embodiments relate to use of a synergistic mixture of fucoxanthin and punicic acid in the manufacture of a medicament for placing cells into a state of hibernation by deacetylating proteins by activating at least one protein selected from the group consisting of Sirt1 and Sirt3 proteins. Various embodiments relate to use of a synergistic mixture of fucoxanthin and punicic acid in the manufacture of a medicament for activating at least one mitochondrial protein selected from the group consisting of Sirt3, Sirt4, and Sirt5.

Various embodiments relate to use of a synergistic mixture of fucoxanthin and punicic acid in the manufacture of a medicament for activating a mitochondrial UCP-1 protein.

Various exemplary embodiments relate to use of a synergistic mixture of fucoxanthin and punicic acid in the manufacture of a medicament for slowing the aging process, and further treating non-alcoholic fatty liver disease, increasing energy expenditure rate, or increasing the volume of brown adipose tissue and/or decreasing the volume of white adipose tissue.

Various exemplary embodiments relate to use of a synergistic mixture in the manufacture of a medicament for slowing the aging process in a mammalian subject by placing cells into a state of hibernation by deacetylating proteins, wherein said synergistic combination comprises fucoxanthin and punicic acid.

Various embodiments relate to a method of improving body composition in a mammalian subject, by activating at least one member of the sirtuin family of proteins, wherein said activating comprises administering to said subject an effective amount of a synergistic combination of fucoxanthin and punicic acid.

Various exemplary embodiments of the fucoxanthin/pomegranate seed oil relate to a composition based on the additive and synergistic actions of its constituents. A first constituent is fucoxanthin. Fucoxanthin may be extracted from, for example, brown marine algae (Undaria pinnatifida, Phaeophyceae). In various exemplary embodiments, such extracts comprise 0.1% by weight to 10% by weight fucoxanthin, preferably 0.5 to 5% by weight, more preferably 0.6 to 1.0% by weight, most preferably 0.8% by weight. In various exemplary embodiments, brown marine algae extracts additionally comprise omega 3-fatty acids. The main fatty acids in brown marine algae are n-3 fatty acids such as ω-3 18:3-α-linolenic acid and ω-3 20:5 eicosapentaenoic acid.

A second constituent of the composition based on the additive is punicic acid. Punicic acid may be included as a pure compound, or punicic acid may be included as a component of pomegranate oil. If punicic acid is included as a component of pomegranate oil, the pomegranate oil typically contains 50-90% punicic acid, preferably 60-80% pomegranate oil, more preferably 70% pomegranate oil. The pomegranate oil is extracted from, for example, pomegranate seed (Punica granatum, Punicaceae). Punicic acid is a 9-cis, 11-trans conjugated linolenic acid (9c,11t,13c-CLNA) and constitutes a major component of pomegranate seed oil.

Various exemplary embodiments of the fucoxanthin/pomegranate seed oil relate to a method of slowing the aging process in a mammalian subject by activating at least one member of the sirtuin family of proteins. Activation of at least one member of the sirtuin family of proteins comprises a step of administering to the subject an effective amount of a synergistic combination of fucoxanthin and punicic acid. The subject may be a human or an animal. The activated member or members of the sirtuin family of proteins may be at least one mitochondrial protein selected from the group consisting of Sirt3, Sirt4, and Sirt5.

Various exemplary embodiments of the fucoxanthin/pomegranate seed oil relate to a method of slowing the aging process in a mammalian subject by activating at least one protein selected from the group consisting of Sirt1 and Sirt3 proteins. Activation of Sirt1 proteins or Sirt3 protein causes deacetylation of proteins, placing cells into a state of hibernation.

Various exemplary embodiments of the fucoxanthin/pomegranate seed oil relate to a method of slowing the aging process in a mammalian subject by activating a mitochondrial UCP-1 protein by administering to the subject an effective amount of a synergistic combination of fucoxanthin and punicic acid.

Various exemplary embodiments of the fucoxanthin/pomegranate seed oil relate to a method of increasing the volume of brown adipose tissue in a subject and decreasing the volume of white adipose tissue by administering to the subject an effective amount of a synergistic combination of fucoxanthin and punicic acid.

Various exemplary embodiments of the fucoxanthin/pomegranate seed oil relate to a method of slowing the aging process in a mammalian subject by activating at least one member of the sirtuin family of proteins by administering to the subject an effective amount of a synergistic combination of fucoxanthin and punicic acid, in combination with at least one omega-3 fatty acid. The omega-3 fatty acid or acids may be derived from brown algae. In certain embodiments, both the fucoxanthin and the omega-3 fatty acid or acids may be derived from extracts of brown algae. In some embodiments, the fucoxanthin may be synthetically produced and the omega-3 fatty acid or acids may be derived from extracts of brown algae. In other embodiments, synthetically produced fucoxanthin may be combined with an extract of brown marine algae, where the extract of brown marine algae contains naturally produced fucoxanthin in combination with omega-3 fatty acids. In various exemplary embodiments of the fucoxanthin/pomegranate seed oil, punicic acid is used as a purified compound. In certain embodiments, punicic acid is added as a component of pomegranate oil. The pomegranate oil contains 50-90% punicic acid, preferably 60-80% pomegranate oil, more preferably 70% pomegranate oil.

In various exemplary embodiments of the fucoxanthin/pomegranate seed oil, the synergistic combination of fucoxanthin and punicic acid comprises:
a) at least one of naturally produced fucoxanthin; synthetically produced fucoxanthin; a brown marine algae extract comprising fucoxanthin; and a brown marine algae extract comprising fucoxanthin and at least one omega-3 fatty acid derived from brown algae;
b) at least one of naturally produced punicic acid; synthetically produced punicic acid; and pomegranate seed oil comprising punicic acid; and
c) optionally at least one omega-3 fatty acid.

Various embodiments disclosed herein relate to a method of improving body composition in a mammalian subject, by activating at least one member of the sirtuin family of proteins, wherein the activating step comprises administering to the subject an effective amount of a synergistic combination of fucoxanthin and punicic acid. In various embodiments, improving body composition includes increasing the volume of brown adipose tissue and decreasing the volume of white adipose tissue in a mammalian subject through treatment with the synergistic combination.

Various embodiments disclosed herein relate to a method of activating Sirt1 in adipose tissue, where said activating may be performed in vitro or in vivo, by contacting the adipose tissue with an effective amount of a synergistic combination of fucoxanthin and punicic acid. In various embodiments, the synergistic composition comprises 50 wt. % of an extract of a brown marine vegetable, said extract comprising 30% by weight of marine vegetable oil, and 0.1% by weight to 10% by weight, preferably 0.5 to 5% by weight, more preferably 0.6 to 1.0% by weight, most preferably 0.8% by weight, fucoxanthin; and 50 wt. % of an pomegranate seed oil, said pomegranate seed oil comprising 60-80% by weight, preferably 70% by weight, of punicic acid.

In various exemplary embodiments of the fucoxanthin/pomegranate seed oil, the synergistic combination of fucoxanthin and punicic acid may be administered by topically applying or parenterally administering the synergistic combination to the subject.

In various exemplary embodiments, an extract of brown marine vegetables containing from 0.1% by weight to 10% by weight fucoxanthin, preferably 0.5 to 5% by weight, more preferably 0.6 to 1.0% by weight, most preferably 0.8% by weight, may be present in an amount from about 25 to 75 weight percent of the composition; and pomegranate seed oil may be present in an amount from about 25 to 75 weight percent of the composition. The extract of brown marine vegetable oil is present in an amount sufficient to provide from 1 mg to 50 mg fucoxanthin per day, preferably 2.0 to 15.0 mg fucoxanthin per day, more preferably 2.0 to 5.0 mg fucoxanthin per day, when taken orally. The pomegranate seed oil is present in an amount sufficient to provide from 1 to 1000 mg punicic acid per day.

In various exemplary embodiments of the fucoxanthin/pomegranate seed oil, the synergistic combination of fucoxanthin and punicic acid may be administered by orally administering the synergistic combination to the subject.

The fucoxanthin/pomegranate seed oil can be administered orally, topically or parenterally, although orally is preferred. The fucoxanthin/pomegranate seed oil is applicable in humans, pet animals and industrial animals.

### BRIEF DESCRIPTION OF THE DRAWINGS

For a fuller understanding of the nature and desired objects of the present fucoxanthin/pomegranate seed oil, reference is made to the following detailed description taken in conjunction with the accompanying drawing figures wherein:

FIG. 1 shows the results of a Western Blot study demonstrating the impact of Xanthigen™, fucoxanthin, and pomegranate seed oil on cellular Sirt1 levels;

FIG. 2A is an HPLC chromatogram of brown marine vegetable extract;

FIG. 2B is an HPLC chromatogram of Fucoxanthin reference standard;

FIG. 3 shows a graph showing the effect of Xanthigen™ on liver fat content in obese subjects with nonalcoholic fatty liver disease (NAFLD) and normal liver fat content (NLF).

### DETAILED DESCRIPTION

In a 16-week, double-blind, randomized, placebo-controlled clinical study, a composition comprising fucoxanthin, omega-3 fatty acids, and pomegranate oil, was evaluated. The fucoxanthin/pomegranate oil composition was compared to fucoxanthin alone, pomegranate seed oil alone, or an olive oil placebo. The daily dietary intake was 1800 kcal (moderate-restricted diet) with no life style modification. Food consumption data, body composition, energy expenditure rate (EER), and blood sample analysis results were assessed on admission and every week for 16 weeks in 110 non-diabetic, obese premenopausal women, including 72 women (n=72) with a liver fat content above 11%—non-alcoholic fatty liver disease (NAFLD), and 38 women (n=38) with a liver fat content below 6.5%—normal liver fat (NLF).

The fucoxanthin/pomegranate seed oil was administered in an amount of 200 mg three-times-a-day (TID) (total daily dosage includes 2.4 mg fucoxanthin and 210 mg pomegranate seed oil) for 16 weeks and resulted in statistically significant reduction of body weight, waist circumference (NAFLD group only), body and the liver fat content, the liver enzymes (NAFLD group only), serum triglycerides and C-reactive protein as compared to the placebo receiving volunteers. The weight loss and reduction in body and liver fat content occurred earlier in the study in patients with NLF than in patients with NAFLD. The fucoxanthin/pomegranate seed oil increased significantly EER in obese subjects as compared to placebo receiving volunteers. Pomegranate seed oil produced an unexpected effect by activating fucoxanthin-induced increase in EER as well as improving the clinical and overall health status of patients as compared to fucoxanthin taken alone.

The disclosed composition increases EER, promotes weight loss, reduces body and specifically liver fat content and improves liver function in obese non-diabetic women. Supplementation is especially beneficial in obese women with NAFLD but also improves health status in women with NLF. Fucoxanthin administered jointly with pomegranate seed oil may be considered a promising food supplement to increase body metabolism, in the management of obesity, and in the normalizing of metabolic and biochemical parameters in the obese subjects.

The disclosed composition contains fucoxanthin, present as a component of brown marine plant extracts, and punicic acid, present as a component of pomegranate seed oil. The composition profoundly increases sirtuin enzyme expression (i.e., Sirt1 expression) in cells. The composition has a substantially greater effect than fucoxanthin alone. The profound increase in Sirt1 expression from a fucoxanthin/punicic acid composition is unexpected since punicic acid alone does not enhance Sirt1 expression at all; in fact, punicic acid suppresses Sirt1 expression.

Activation of Sirt1 by a fucoxanthin/punicic acid composition impacts the body in a number of ways. First, activation of sirt1 induces expression of UCP1. While fucoxanthin has recognized weight loss properties based on the induction of the UCP1, combination of fucoxanthin with pomegranate seed oil greatly enhances Sirt1 activation, and hence enhances UCP1 expression more strongly than fucoxanthin alone. The enhancement of UCP1 by a fucoxanthin/punicic acid composition also leads to an increased energy expenditure rate (EER) by uncoupling a step in cellular metabolism. Fucoxanthin/punicic acid also enhances Sirt3 expression in brown adipose tissue (BAT), and the deacetylase activity of Sirt3 induces increased levels of BAT and its signature uncoupling protein-1.

Fucoxanthin, alone or in combination with punicic acid, up-regulates expression of the UCP1 gene in WAT, contributing to the reduction of white adipose tissue and a significant reduction of body weight in KK-Ay mice. Fucoxanthin, alone or in combination with punicic acid, also suppresses adipocyte differentiation and lipid accumulation, thereby inhibiting glycerol-3-phosphate dehydrogenase activity. Further, fucoxanthin/punicic acid also enhances Sirt3 expression in brown adipose tissue (BAT), and the deacetylase activity of Sirt3 induces increased levels of BAT and its signature uncoupling protein-1.

Glycerol-3-phosphate dehydrogenase has been linked to body mass index, WAT, and blood glucose Glycerol-3-phosphate dehydrogenase knockout mice were found to have a lower body mass index, a 40% reduction in the weight of WAT, and lower fasting blood glucose, as compared to the matching control. Additional hypolipidemic properties of fucoxanthin come from down-regulating peroxisome proliferator-activated receptor-γ (PPART-γ), responsible for adipogenic gene expression.

Dietary pomegranate seed oil significantly reduces serum TG levels, a predominant body fat contributing to adiposity and WAT. Punicic acid of pomegranate has been shown to suppress delta-9 desaturase (an enzyme in fat metabolism), a possible mechanism behind the effect of pomegranate seed oil in lowering the hepatic TG accumulation. The brown algae omega-3 fatty acids may provide an additional mechanism to decrease serum and liver TG concentrations due to the reported omega-3 promotion of hepatic fatty acid β-oxidation. This hypolipidemic mechanism may be further potentiated by co-administration of fucoxanthin with omega-3 fatty acids, which can increase the amounts of dietary omega-3 fatty acids in the liver.

The principal mechanism of the disclosed composition is due to its unexpected effect on energy expenditure rate (EER) and the related metabolic and clinical effects as compared to fucoxanthin or pomegranate oil used alone. Energy expenditure rate (EER) was measured in patients by indirect calorimetry. Oxygen was measured with an electrochemical oxygen sensor, and carbon dioxide was measured with an infrared carbon dioxide sensor (Ametec Carbon Dioxide Analyzer). Before each measurement, the instrument was calibrated with a mixture of O₂ and CO₂ gases. Rates of oxygen consumption (O₂) and carbon dioxide production (CO₂) were calculated and printed out at 1-min intervals. Energy expenditure was derived from O₂ and CO₂ values.

Supplementation with the disclosed fucoxanthin/pomegranate seed oil composition as compared to supplementation with fucoxanthin or pomegranate seed oil/punicic acid alone increased EER, especially in obese subjects with non-alcoholic fatty liver disease or NAFLD, but also in subjects with normal fat liver content or NFL. The minimum effective dose of fucoxanthin stand alone was 2.4 mg. A lower fucoxanthin dose of 1.6 mg, although not effective per se, when supplemented with pomegranate seed oil (200 mg per day) showed an unexpected significant increase in EER (p<0.05) and other clinical benefits. Thus, administration of two 200 mg capsules, each comprising 0.8 mg fucoxanthic and 100 mg pomegranate seed oil, is effective. The EER was further increased with the higher dose of the pomegranate seed oil. These results suggest that pomegranate seed oil has triggering and synergistic dose dependent effects on fucoxanthin-induced increase in EER. The pomegranate seed oil/punicic acid stand alone administration had no effect on EER.

The clinical study of the fucoxanthin/pomegranate seed oil composition demonstrates that pomegranate oil acts synergistically in combination with fucoxanthin to produce the EER-stimulating action of fucoxanthin. As seen in Table 4 (discussed in further detail below), fucoxanthin alone significantly increases energy expenditure rate, while pomegranate oil alone has little or no effect on energy expenditure rate. However, the combination of fucoxanthin and pomegranate oil increases energy expenditure rate substantially more than fucoxanthin alone. The synergistic fucoxanthin/pomegranate oil combination also results in increased metabolic rate, WAT loss, and body weight-loss. The synergistic fucoxanthin/pomegranate oil combination also produces reduced waist-hip ratio (WHR) and normalizes of homeostatic functions of the body, as attested by a normalized blood pressure and indices of inflammation, e.g. c-reactive protein or CRP. The clinical study provides evidence of activation of Sirt1-7 proteins which would sustain body composition and metabolic functions gains by promoting BAT and also slow down the accumulation of WAT with the aging process. The expression of UCP-1 protein may be potentiated or attenuated by the fucoxanthin/pomegranate seed oil composition which would reflect on the impact of the composition on BAT biogenesis and activation of Sirtuin proteins.

As previously mentioned, the obese patients with NAFLD and also with those with NFL benefited from the fucoxanthin/pomegranate seed oil composition with decrease in liver and visceral fat, decrease in plasma oxidized LDL, and decrease in inflammatory processes in the body, e.g. decrease in serum CRP levels, which positively correlates with development of insulin-resistance, metabolic syndrome and diabetes type 2.

The fucoxanthin/pomegranate seed oil composition provides a nutrigenomic approach to a complex metabolic disorder with effects on the genome, epigenome, and proteome of the organism. This multiple nutrigenomic mechanism prevents the common recurrence of excess body fat (yo-yo effect) securing resilience to obesity. The fucoxanthin/pomegranate seed oil composition regulates BAT and WAT and adipokines including leptin, adiponectin, visfatin, resistin, interleukin (IL)-6 and TNF which in turn influence energy metabolism, insulin sensitivity, cardiovascular health and overall health. With its broad mechanism of action, the fucoxanthin/pomegranate seed oil composition has organ-specific effect of decreasing liver triglycerides content.

The observed normotensive effect of the fucoxanthin/pomegranate seed oil composition, which may be symptomatic of its broad homeostatic mechanism in obese individuals, is due to a significant reduction in body weight, the body and liver fat content, reduction in serum TG, markers of inflammation and the liver enzymes. Adiponectin is a WAT adipocyte-derived cytokine which acts in the CNS to control autonomic function, energy, and cardiovascular homeostasis, resulting in the normotensive effects in the study population receiving the fucoxanthin/pomegranate seed oil composition. The fucoxanthin/pomegranate seed oil composition stimulates adiponectin, which exerts homeostatic effects seen in the clinical study.

The levels of another adipokine generated by adipocytes, leptin, are decreased as a result of the mechanism of the fucoxanthin/pomegranate seed oil composition. The increased adiponectin to leptin ratio as a result of the fucoxanthin/pomegranate seed oil composition correlates with weight loss, and increase in BAT to WAT ratio and improved and economized metabolic energy processes. It has been shown that the presence of specific fatty acids in fasting plasma could have a significant impact on the level of inflammatory markers. For example, lower α-linolenic acid content is associated with higher CRP, whereas a high plasma n-3 fatty acid content is associated with lower levels of pro-inflammatory and higher levels of anti-inflammatory markers. Although punicic acid (PA) is structurally related to linolenic and linoleic acids, it has a distinct mechanism of action. For example, the animals on diets with conjugated linoleic acid or a mixture of conjugated linolenic acid isomers other than PA resulted in development of insulin resistance and fatty liver despite a significant decrease in body weight. The individuals taking the fucoxanthin/pomegranate seed oil composition are losing WAT, which effect may involve proteins participating in the triglycerides metabolism, i.e. perilipins in adipocytes, which activity is linked with altered activity of UCP1 and increased BAT.

The perilipin gene codes for perilipin, a protein that coats intracellular lipid droplets and modulates lipolysis in adipocytes. The perilipin (PAT) family of lipid droplet proteins includes 5 members in mammals: perilipin, adipose differentiation-related protein (ADRP), tail-interacting protein of 47 kDa (TIP47), S3-12, and OXPAT. Perilipins are present in evolutionarily distant organisms, including insects, slime molds and fungi. These proteins are similar in structure and the ability to bind intracellular lipid droplets, either constitutively or in response to metabolic stimuli, e.g. increased lipid flux into or out of lipid droplets. Positioned at the lipid droplet surface, perilipins manage the access to other proteins (lipases) and also to the lipid esters within the lipid droplet core. Perilipins can interact with the cellular machinery important for lipid droplet biogenesis. The importance of perilipin as modulator of lipolysis is underscored by published studies demonstrating that over-expression of perilipin in adipocytes results in decreased lipolysis as well as perilipin-knockout mice showing evidence of increased levels of basal lipolysis and obesity-resistance. The dephosphorylated form of the perilipin protein restricts access to lipid droplets, preventing lipid mobilization. When hormones signal the need for metabolic energy, lipids must be brought out of storage and transported to tissues where fatty acids can be oxidized for energy production. To this end, the enzyme adenylate cyclase is activated and in turn leads to cAMP-dependent protein kinase (PKA) phosphorylation of perilipin. The phosphorylated form of perilipin allows lipases in the cytosol to move to the lipid droplet and hydrolyze TG to free fatty acids and glycerol.

The present fucoxanthin/pomegranate seed oil composition has broad metabolic activity due to the nutrigenomic activation of the Sirt1-7 cascade, decreasing WAT in favor of BAT, improving energy expenditure rate, improving glucose tolerance, decreasing markers of inflammation, lowering blood pressure, decreasing body weight, and improving the overall health status of the individual. As discussed above, sirtuin enzymes, e.g. Sirt1 and Sirt3, exert their function by removing the acetyl group from proteins. The deacetylation results in inactivation of the proteins' role in cell metabolism and prevents genes from over-expression, thereby putting a cell into a state of hibernation and increasing its lifespan. The fucoxanthin/pomegranate seed oil composition increases resilience to obesity by slowing down primary aging (increasing longevity), and exerting a protective effect against secondary aging by decreasing the incidence of chronic degenerative diseases.

Collectively, the fucoxanthin/pomegranate seed oil composition resembles effects of a caloric restriction (CR) diet. However, it is unlikely that most humans would be willing to maintain a 30% reduced diet for the bulk of their adult life span, even if it meant more healthy years. For this reason, the fucoxanthin/pomegranate seed oil composition is particularly useful as CR mimetic providing the same beneficial effects as CR, without the necessity of excessive dieting. Without requiring a dramatically lower food intake, the fucoxanthin/pomegranate seed oil composition favorably affects immune functions and hormonal profiles, especially those that reduce glucose/energy flux

Without being bound by any theory, the present inventors believe that the fucoxanthin/pomegranate seed oil composition increases resistance to the aging process by exerting the broad regulatory homeostatic mechanism. Insulin resistance is one of the important reasons for increasing carbohydrate metabolism malfunction with aging. The mechanisms of these changes have been partially elucidated. Decreased physical activity and increased total, and, specifically, abdominal and liver fat are especially important pathogenetic mechanisms.

Changes in the glucose transporter 4 (GLUT-4) level in skeletal muscles and the serum level of insulin-like growth factor 1 (IGF-1) could be mechanisms independent of fat tissue. Other mechanisms which could be associated with insulin resistance in aging are related to leptin and adiponectin serum levels or changes in mitochondrial energy metabolism and levels of advanced glucose end products in diet.

Many studies suggest the decrease of beta cell function in people older than 60 years. Age-associated defects of beta cell function can be detected in loading tests, especially with prolonged intravenous glucose infusion. The defective mechanisms are abnormalities in insulin processing, insulin secretion, insulin release kinetics with parallel lower insulin secretion capacity, the impossibility of increasing insulin release properly, and age-increasing insulin resistance. These pathogenetic changes are related to the increase of visceral fat deposits and other mechanisms such as defects in beta cell structure, decrease in glucose and incretins sensing and the defective course of replication/neogenesis processes. In addition, the possibility of exploiting the plasticity of the adipose organ, with conversion of white adipocytes in white adipose tissue to atypical brown adipocytes and increasing thermogenesis in them is considered as another potential target for increasing energy expenditure in humans.

### Example 1

### The Effect of Fucoxanthin/Punicic Acid Mixtures on Sirt1 Expression

### Cell Culture

The effect of fucoxanthin/punicic acid mixtures on Sirt1 expression in murine cells was studied. Mouse 3T3-L1 pre-adipocytes purchased from the American Type Culture Collection (Rockville, Md.) were grown in Dubecco's modified Eagle's medium (DMEM) (Gibco BRL, Grand Island, N.Y.) supplemented with 2 mM glutamine (GIBCO BRL), 1% penicillin/streptomycin (10000 units of penicillin/mL and 10 mg streptomycin/mL) and 10% fetal bovine serum at 37° C. under a humidified 5% CO₂ atmosphere.

For differentiation of 3T3-L1 pre-adipocytes, cells were seeded into 6-well culture plates (2×104/mL) and cultured as described above. Two days after confluence (defined as day 0), cells were incubated in differentiation medium containing 1.7 μM insulin, 0.5 mM 3-isobutylmethylxanthine (IBMX) and 12.7 μM dexamethasone (DEX) in DMEM containing 10% fetal bovine serum (FBS) for 48 h. The medium was then replaced by DMEM containing 10% FBS and insulin (1.7 μM) with or without (10, 50 and 100 μg/mL) Fucoxanthin extract, Xanthigen™ or pomegranate seed oil, and changed to fresh medium every two days. After 12 days, the cells were harvested and then total protein was extracted for Western Blot analysis.

The fucoxanthin extract used in this study of Sirt1 expression in murine cells was an extract of the complete plant of Undaria pinnatifica. The extract contains 0.8% by weight fucoxanthin and 30% by weight marine vegetable oil. The fatty acid compositions of fucoxanthin extract is shown in Table 2. The fucoxanthin extract used may further contain ≦10.0 wt. % palm oil. The pomegranate seed oil used in this study contains 70 wt. % punicic acid, and is an extract of the seeds of the Punica granatum plant. Xanthigen™, the fucoxanthin/punicic acid mixture used in this study, contains 50 wt. % of the fucoxanthin extract, and 50 wt. % of the pomegranate seed oil.

Western Blot analysis was carried out by extracting the total proteins via addition of 100 μL of gold lysis buffer (50 mM Tris-HCl, pH 7.4; 1 mM NaF; 150 mM NaCl; 1 mM EGTA; 1 mM phenylmethanesulfonyl fluoride; 1% NP-40; and 10 μg/mL leupeptin) to the cell pellets on ice for 30 min, followed by centrifugation at 10,000×g for 30 min at 4^{L}. The total proteins are measured by Bio-Rad Protein Assay (Bio-Rad Laboratories, Munich, Germany). The samples (50 μg of protein) were mixed with 5× sample buffer containing 0.3M Tris-HCl (pH 6.8), 25% 2-mercaptoethanol, 12% sodium dodecyl sulfate (SDS), 25 mM EDTA, 20% glycerol, and 0.1% bromophenol blue. The mixtures were boiled at 100° C. for 5 min and subjected to 10% SDS-polyacrylamide minigels at a constant current of 20 mA. Following electrophoresis, proteins on the gel were electrotransferred onto an immobile membrane (PVDF; Millipore Corp., Bedford, Mass.) with transfer buffer composed of 25 mM Tris-HCl (pH8.9), 192 mM glycine, and 20% methanol. The membranes were blocked with blocking solution containing 20 mM Tris-HCl, and then immunoblotted with primary antibodies including antibodies to Sirt1 and β-actin (Transduction Laboratories, Lexington, Ky.). The blots are rinsed three times with PBST buffer for 10 min each. Then blots are incubated with 1:5000 dilution of a horseradish peroxide (HRP)-conjugated secondary antibody (Zymed Laboratories, San Francisco, Calif.) and then washed again three times with PBST buffer. The transferred proteins were visualized with an enhanced chemiluminescence detection kit (ECL; Amersham Pharmacia Biotech, Buckinghamshire, UK).

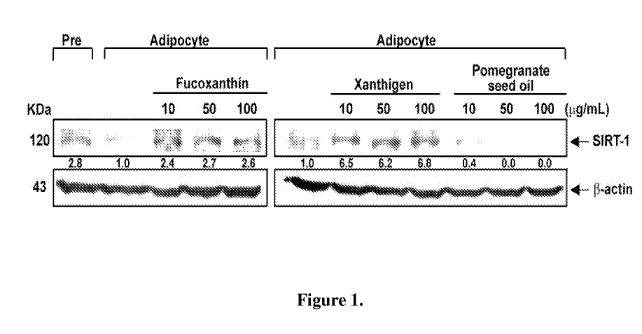

As shown in FIG. 1, the protein expression of Sirt1 in differentiated 3T3-L1 adipocytes was notably decreased compared with 3T3-L1 pre-adipocytes. On the other hand, treatment with Fucoxanthin extract and Xanthigen™ markedly increased Sirt1 protein levels in differentiated 3T3-L1 adipocytes, whereas treated pomegranate seed oil was no effect, as seen in Table 1.

A comparison of the Western Blot results for 3T3-L1 pre-adipocytes and differentiated adipocytes shows that the levels of Sirt1 in 3T3-L1 pre-adipocytes are about 280% of Sirt1 levels in differentiated adipocytes (2.8 vs. 1.0), in the absence of added xanthigen, fucoxanthin, or pomegranate seed oil, as shown in Table 1. Differentiated 3T3-L1 adipocytes in the absence of added Xanthigen™, fucoxanthin, or pomegranate seed oil are used as controls in these experiments.

The Western Blot results for differentiated 3T3-L1 adipocytes which have been treated with fucoxanthin show that fucoxanthin increases Sirt1 levels in differentiated adipocytes to nearly the levels of Sirt1 levels in pre-adipocytes (2.4-2.7 vs. 2.8). The amount of the increase is independent of dose (10 micrograms/ml, 50 micrograms/ml, and 100 micrograms/ml give similar results). The Western Blot results for differentiated 3T3-L1 adipocytes which have been treated with pomegranate seed oil are very different from the results seen with fucoxanthin. Treatment with pomegranate seed oil actually suppresses the levels of Sirt1 in differentiated 3T3-L1 adipocytes, relative to Sirt1 levels in the control group (0.0-0.4 vs. 1.8). The amount of the increase appears to be dose-dependent (some Sirt1 activity is seen in cells treated with 10 micrograms/ml pomegranate seed oil, but no Sirt1 activity is seen in cells treated with 50-100 micrograms/ml pomegranate seed oil). Therefore, fucoxanthin and pomegranate seed oil have opposite effects on Sirt1 activation in differentiated cells.

Treatment with a mixture comprising 50 wt. % of an Undaria pinnatifica extract containing 0.8 wt. % fucoxanthin and 50 wt. % of pomegranate seed oil (Xanthigen) greatly enhanced Sirt1 levels in differentiated 3T3-L1 adipocytes. Sirt1 levels in differentiated adipocytes treated with Xanthigen have been increased to between 220-243% of the levels of Sirt1 levels in pre-adipocytes (6.2-6.8 vs. 2.8), and over six times Sirt1 levels in the control group. The amount of the increase is independent of dose (10 micrograms/ml, 50 micrograms/ml, and 100 micrograms/ml give similar results).

Therefore, even though fucoxanthin and pomegranate seed oil have opposite effects on Sirt1 activation in differentiated cells, their combination produces greater Sirt1 activation than fucoxanthin alone. This result is unexpected because pomegranate seed oil alone does not enhance Sirt1 levels, but rather suppresses Sirt1 activation.

TABLE 1
The effect of fucoxanthin/punicic acid mixtures
on Sirt1 expression.

| | | Sirt1 expression |
|---|---|---|
| Cells | Treatment (μg/mL) | (% of control) |
| 3T3-L1 pre- | None | 280% |
| adipocytes | | |
| Differentiated | None | 100% |
| adipocytes | (control) | |
| Differentiated | fucoxanthin extract* (10) | 240% |
| adipocytes | | |
| Differentiated | fucoxanthin extract (50) | 270% |
| adipocytes | | |
| Differentiated | fucoxanthin extract (100) | 260% |
| adipocytes | | |
| Differentiated | pomegranate seed oil (10) | 40% |
| adipocytes | | |
| Differentiated | pomegranate seed oil (50) | 0% |
| adipocytes | | |
| Differentiated | pomegranate seed oil (100) | 0% |
| adipocytes | | |
| Differentiated | fucoxanthin/ | 650% |
| adipocytes | pomegranate seed oil** (10) | |
| Differentiated | fucoxanthin/ | 620% |
| adipocytes | pomegranate seed oil (50) | |
| Differentiated | fucoxanthin/ | 680% |
| adipocytes | pomegranate seed oil (100) | |

*Fucoxanthin extract contains 50 wt. % of a Undaria pinnatifica extract containing 0.8 wt. % fucoxanthin.
**Fucoxanthin/pomegranate seed oil contains 50 wt. % of an Undaria pinnatifica extract containing 0.8 wt. % fucoxanthin; and 50 wt. % of pomegranate seed oil.

### Example 2

### The Effect of Different Dose of Experimental Sample on the Energy Expenditure on Obese Subjects

Obese subjects diagnosed with NAFLD and with apparently healthy liver (HL) were matched in pairs based on age, body weight and body fat mass and were randomly divided into Experimental NAFLD group (n=36), Placebo NAFLD (n=36), Experimental HL (n=19) and Placebo-HL group (n=19). Subjects were randomly assigned, in equal numbers, to the phytomedicine experimental groups and the Placebo control group, using the Simple Randomization Procedure. Their daily dietary intake was restricted to 1800±100 kcals, of which 50±5% was in the form of carbohydrates, 30±5% from protein, and 20±5% from fat. Subjects were also instructed to consume all the foods and beverages designated by dieticians and provided by the Institute, and to eat no other food or high calories beverages. Patients were directed to take Experimental Sample and/or Placebo three times a day before meals. During the clinical phase, subjects were required to visit a designated hospital three times a week for physiological and biochemical analysis. Institute provided all foods and beverages by designated dieticians and labeled as B, L, and D for breakfast, lunch and dinner, respectively.

Food record analysis, body composition, blood and adipose biopsy samples were assessed throughout the trial. All volunteers underwent medical examination, including laboratory testing of serum alanine aminotransferase (ALT), aspartate aminotransferase (AST), -glutamyltransferase (GGT) enzymes activity. All participants had negative serology for hepatitis B or C. Subjects taking medications known to influence fat metabolism were also excluded.

### Oral Glucose Tolerance Test

To exclude obese subjects with diabetes, standard oral glucose tolerance test with 75 g of glucose was performed as described previously. The absence of clinically manifested diabetes criteria was also included during selection procedure.

### Experimental Sample

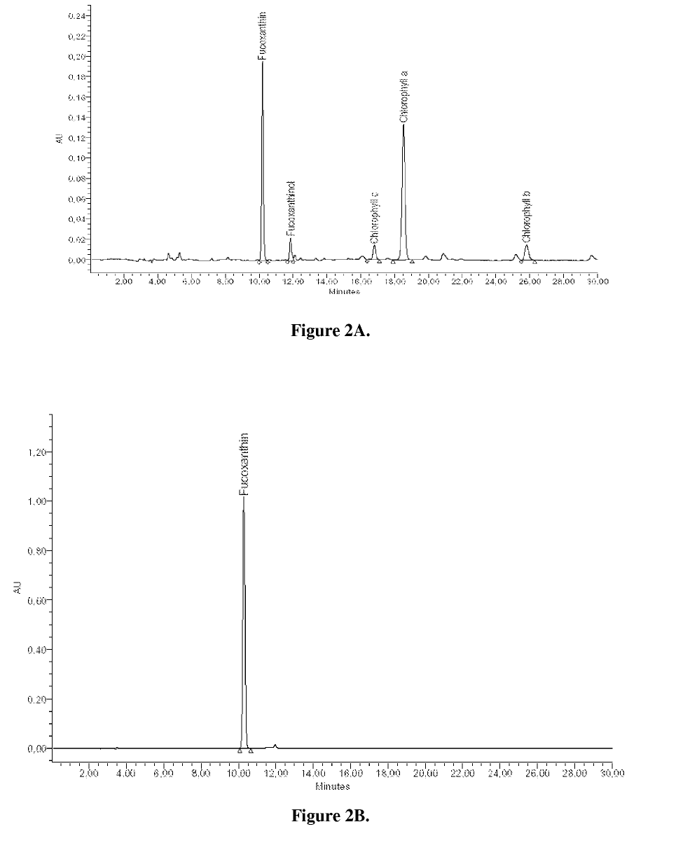

Each capsule of experimental supplement sample used in our clinical trial was prepared from 100 mg of a brown marine vegetable extract containing 0.8% by weight fucoxanthin (0.8 mg fucoxanthin per capsule) and 30 mg marine vegetable oil. The brown marine vegetable extract was suspended in 100 mg cold-pressed pomegranate seed oil. The pomegranate seed oil was standardized to contain a minimum of 70% punicic acid, for a total weight of 200 mg/capsule. The content of fucoxanthin in Experimental Sample (Xanthigen) was analyzed using high performance liquid chromatography method, and the fatty acids were analyzed by Gas Chromatography method. The HPLC profile of the brown marine vegetable extract is shown in FIG. 2A, along with an HPLC chromatogram of pure fucoxanthin for comparison in FIG. 2B. The fatty acid compositions of brown marine vegetable extract and cold-pressed pomegranate seed oil are shown in Table 2.

TABLE 2
Typical fatty acid composition of brown marine vegetable extract
and cold-pressed pomegranate seed oil

| | Brown marine | Pomegranate |
|---|---|---|
| | vegetable | seed oil |
| Fatty acid | (30% w/w) | (90% w/w) |
| 14:0 Myristic acid | 2.8 | — |
| 16:0 palmitic acid | 14.9 | 2.4 |
| 16:1 palmitoleic acid | 6.4 | 0.3 |
| 18:0stearic acid | — | 1.2 |
| 18:1 oleic acid ω-9 | 4.5 | 5.9 |
| 18:2 linoleic acid ω-6 | 4.5 | 9.2 |
| 18:3-γ linolenic acid ω-6 | — | — |
| 18:3-α-linolenic acid ω-3 | 12.1 | 0.2 |
| 18:3 triple conjugated (Punicic acid) | — | 80.8 |
| 18:4 stearidonic acid ω-3 | 26.7 | — |
| 20:4 arachidonic acid ω-6 | 11.8 | — |
| 20:5 eicosapentaenoic acid ω-3 | 16.3 | — |

### Total Weight and Body Fat Analysis

The body weight and fat mass index and visceral fat were evaluated. A total body scan was performed using dual-energy X-ray absorptiometry to determine percent body fat, lean body mass and fat mass. Fat-free mass and fat mass were calculated by the equations developed from a study using the four-compartment model on a cohort by Heitmann (1990). Height was measured to the nearest 0.5 cm and body weight to the nearest 25 g. Subjects were wearing light clothes and circumferences were taken to the nearest 0.5 cm.

### Measurements of Fat Oxidation by Indirect Calorimetry

Energy expenditure (EE) and substrate oxidations were measured by indirect calorimetry as described previously (Ranneries et al., 1998). Oxygen was measured with an electrochemical oxygen sensor, and carbon dioxide was measured by an infrared carbon dioxide sensor (Ametec Carbon Dioxide Analyzer). Calculations of EE and substrate oxidation rates were performed as previously described (Astrup et al., 1991). Protein oxidation was assumed to be constant and amounting to 15% of EE. The error of calculating EE by omitting the exact correction from urinary nitrogen was negligible and impossible to estimate during such a short period of time. The reliability was assessed by the coefficient of variation on resting energy expenditure repeated every week.

After all subjects completed 16 weeks clinical trial, no adverse effect in both groups throughout the trial occurred and no evidence for increase in blood pressure or cardiac disturbances was obtained. Subjects tolerated phytomedicine Experimental supplement sample and the placebo, as well as foods designed by professional dietician and provided by the designated Hospital. The physical and anthropometrical characteristics of the subjects are given in Table 3. There was no significant difference between the two groups for any of the measurements.

TABLE 3
Physical and anthropometrical characteristics
of subjects participated in the trial

| Variable | n = 41 |
|---|---|
| Age, yr | 37.4 ± 4.8 |
| Body weight, kg | 91.5 ± 4.4 |
| Body Fat, kg | 40.4 ± 3.7 |
| Liver Fat, % | 15.1 ± 2.9 |
| CRP, mg/L | 6.8 ± 3.7 |
| ALT units/L | 49 ± 11 |
| AST units/L | 50 ± 9 |
| GGT units/L | 46 ± 8 |
| Systolic Blood Pressure | 130 ± 8 |
| (mmHg) | |
| Diastolic Blood Pressure | 87 ± 7 |
| (mmHg) | |

Results of this clinical study, as shown in Table 4, indicated that the supplementation of Experimental Sample (Xanthigen) stimulated daily energy expenditure in obese subjects. This effect was clearly dose-dependent phenomenon. No statistically significant increase in the energy expenditure was observed in subjects who received 200 and 400 mg of Experimental Sample per day, while dramatic increase in the energy expenditure rates was observed in obese subjects who received 600 mg and 1000 mg of Experimental Sample per day, where 600 mg of Experimental Sample corresponds to 15 mg fucoxanthin and 1000 mg of Experimental Sample corresponds to 25 mg fucoxanthin. As seen in Table 4,600 mg of Experimental Sample results in an increase in daily energy expenditure rate of 1670±310 kJ/day, which is substantially greater than that achieved with 25 mg fucoxanthin alone (1152±290 kJ/day); 600 mg of Experimental Sample also results in a change in energy expenditure rate which is vastly greater than that obtained with 600 mg placebo (58±40 kJ/day) or with 1500 mg pomegranate oil alone (159±65 kJ/day). Based on our dose-response trial, the optimum dose of Experimental Sample was established as 600 mg per day, which was used in further clinical trials.

TABLE 4
Effect of Xanthigen ™, fucoxanthin, pomegranate seed oil and olive oil
on Energy Expenditure rates in obese non-diabetic female volunteers with NAFLD.

| | | | | | | Change in Total | a.P value as |
|---|---|---|---|---|---|---|---|
| Dosage per day, | | | | | | Energy Expenditure | compared |
| n = number of subjects | Baseline* | 2 weeks* | 5 weeks* | 10 weeks* | 16 weeks* | (kJ/24 h) | to placebo |
| 600 mg Placebo, | 5.91 ± 0.32 | 5.95 ± 0.26 | 5.55 ± 0.24 | 5.59 ± 0.32 | 5.95 ± 0.19 | From 8510 to 8568, | |
| n = 3 (Olive oil) | | | | | | Net 58 ± 40 | |
| Xanthigen ™-200 mg | 5.72 ± 0.22 | 5.54 ± 0.32 | 5.59 ± 0.29 | 5.67 ± 0.36 | 5.88 ± 0.31 | From 8237 to 8467, | P < NS** |
| n = 3; 5 mg Fucoxanthin | | | | | | Net 230 ± 125 | |
| Xanthigen ™-400 mg | 6.02 ± 0.17 | 5.98 ± 0.29 | 6.12 ± 0.31 | 6.53 ± 0.15 | 6.43 ± 0.22 | From 8668 to 9259, | p < 0.05 |
| n = 3; 10 mg Fucoxanthin | | | | | | Net 591 ± 210 | |
| Xanthigen ™-600 mg | 5.87 ± 0.30 | 5.68 ± 0.52 | 6.43 ± 0.43 | 6.88 ± 0.27 | 7.03 ± 0.33 | From 8453 to 10123, | P < 0.05 |
| n = 4; 15 mg Fucoxanthin | | | | | | Net 1670 ± 310 | |
| Xanthigen ™-1000 mg, | 5.92 ± 0.12 | 6.11 ± 0.30 | 6.47 ± 0.33 | 6.79 ± 0.21 | 7.09 ± 0.28 | From 8524 to 10210, | p < 0.05 |
| n = 4; 25 mg Fucoxanthin | | | | | | Net 1686 ± 290 | |
| Fucoxanthin, | 5.82 ± 0.18 | 5.69 ± 0.21 | 5.93 ± 0.15 | 5.69 ± 0.21 | 5.98 ± 0.18 | From 8381 to 8611, | p < NS** |
| n = 4; 10 mg | | | | | | Net 230 ± 147 | |
| Fucoxanthin, | 5.85 ± 0.27 | 5.89 ± 0.14 | 5.98 ± 0.17 | 6.11 ± 0.21 | 6.39 ± 0.17 | From 8424 to 9202, | p < 0.05 |
| n = 4; 15 mg | | | | | | Net 778 ± 260 | |
| Fucoxanthin, | 5.92 ± 0.16 | 6.02 ± 0.23 | 5.92 ± 0.27 | 6.29 ± 0.31 | 6.72 ± 0.22 | From 8525 to 9677, | p < 0.05 |
| n = 4; 25 mg | | | | | | Net 1152 ± 290 | |
| Fucoxanthin, | 6.04 ± 0.24 | 5.91 ± 0.31 | 6.32 ± 0.22 | 6.92 ± 0.31 | 7.37 ± 0.35 | From 8698 to 10613, | p < 0.001 |
| n = 4; 50 mg | | | | | | Net 1915 ± 246 | |
| Pomegranate Seed Oil, | 6.01 ± 0.19 | 5.89 ± 0.32 | 5.92 ± 0.27 | 6.02 ± 0.19 | 6.12 ± 0.24 | From 8654 to 8813, | p < NS** |
| n = 4; 1500 mg | | | | | | Net 159 ± 65 | |
| Pomegranate Seed Oil, | 5.95 ± 0.24 | 6.00 ± 0.30 | 6.10 ± 0.26 | 6.02 ± 0.25 | 6.07 ± 0.19 | From 8568 to 8741, | p < NS** |
| n = 4; 2000 mg | | | | | | Net 173 ± 92 | |

*Energy Expenditure measured in kJ/min.
**NS = Not Significant

### Example 3

### Effect of Experimental Sample on Plasma Serum Enzyme and Liver Fat and in Obese Subjects with NAFLD

AST, ALT, and GGT are sensitive indicators of liver cell injury, and have been used to identify patients with liver disease for almost 50 years. Elevated serum ALT, ALT and GGT levels help identify many types of liver diseases in patients and have widely used to screen blood donors for non-A, non-B hepatitis. Any type of liver cell injury can modestly increase ALT, ALT and GGT levels. High plasma ALT is associated with decreased hepatic insulin sensitivity and predicts the development of type 2 diabetes (Vozarova et al., 2002). Marked elevations of these enzymes occur most often in persons with diseases that affect primarily hepatocytes, such as viral hepatitis, ischemic liver injury (shock liver), and toxin-induced liver damage. Currently, measurement of serum ALT, AST and GGT levels is the most frequently used test to identify patients with liver diseases. The levels of plasma ALT, AST and GGT are correlated strongly with BMI, obesity, and with fatty liver (NAFLD).

Patients with NAFLD are commonly characterized by elevated concentrations of markers of liver injury, including AST, ALT and GGT (Mulhall et al., 2002; Angulo, 2002). Furthermore, NAFLD has been reported to be the most common cause of chronically elevated aminotransferase levels (Clark et al., 2003). These observations indicate that AST, ALT, and other markers of liver injury may be useful surrogate measures of NAFLD and related conditions for large studies. The purpose of this study was to investigate the effects of novel dietary supplement on the levels of plasma marker inflammation enzymes AST, ALT and GGT, and liver fat in the obese subjects with NAFLD.

### Subjects

Seventy two (n=72) obese pre-menopausal female subjects, with an average body weight of 94.5±2.1 and average age of 34±3.5 years were recruited to take part in a double-blind, placebo-controlled, randomized clinical trial. All volunteers underwent medical examination, including laboratory testing of ALT, AST and GGT enzymes activity. All participants had negative serology for hepatitis B or C. Subjects taking medications known to influence fat metabolism were also excluded.

### Liver Fat Analysis

Subjects with apparently healthy liver and NAFLD were screened and subjected to magnetic hepatic ultrasound scanning by professional physicians using Acuson 128-XP/10 scanner with a 3.5-MHz linear transducer, according to the conventional criteria. In addition, we used complementary method described previously (Thomsen et al., 1994) and Image-guided proton magnetic resonance spectroscopy method (Magnetom Vision, Siemens, Erlangen, Germany) described in details elsewhere (Seppälä-Lindroos et al., 2002). The percent liver fat was calculated by dividing 100 times Sfat by the sum of Sfat and Swater (Ryysy et al., 2000).

From 140 obese subjects evaluated for NAFLD, 96 subjects were diagnosed positive for fatty liver disease. The selection criteria for NAFLD were the content of liver fat higher than 14±4%.

### Experimental Supplement Sample

Each soft-gel capsules of Experimental supplement sample used in this clinical trial and placebo was prepared by Center of Modern Medicine, Institute of Immunopathology, Moscow, using the method described above.

### Blood and Urine Samples Collection

Venous blood and urine samples were collected into tubes containing sodium EDTA (1 g/L). Blood samples were collected once a week in the morning during 16 weeks of the trial. Plasma samples were prepared within 1 hour after the blood collection by centrifugation at 600×g for 15 min at 4° C. Blood samples were kept in the dark and on ice until centrifugation. Plasma samples were immediately divided into aliquots and stored under argon at −70° C. Urine samples were collected at the beginning and at the end of the clinical trial. The volumes of the collected urine samples were measured and aliquots were stored at −20° C.

### Serum Enzyme Analysis

Venous blood was drawn in the morning after an overnight fast. At baseline and throughout trial serum enzymes AST, ALT, and GGT activity were analyzed using the methods published in Standard Laboratory Manual. Metabolic syndrome was defined according to criteria proposed by the National Cholesterol Education Program Adult Treatment Panel III (ATP III) (Jousilahti et al., 2000; Lee et al., 2003; Perry et al., 1998; Nakanishi et al., 2003.)

### Results

Patients well tolerated 600 mg Experimental Sample and no sign of adverse toxic effects was observed. Statistically significant reduction of ALT, AST and GGT was observed after 16 weeks of supplementation of Experimental Sample in all subjects. The levels of plasma ALT were reduced from its baseline 51±9 units/L to 26±7 units/L (p<0.005), plasma AST levels reduced from 53±7 units/L to 29±6 units/L (p<0.005) and GGT from 49±5 units/L to 31±5 units/L (p<0.005). Furthermore, the level of these enzymes persisted in normal range 2 weeks after with-drove period.

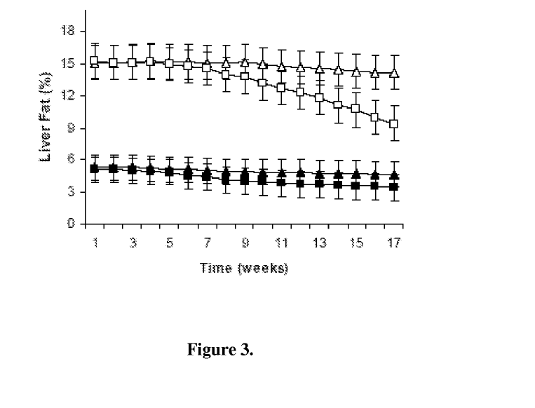

The reduction of plasma ALT, AST, GGT levels were correlated with significant reduction of liver fat. Statistically significant reduction of liver fat was observed after 16 weeks of Experimental Sample supplementation. The content of the liver fat was reduced from 15.3±4.1% to 9.4±3.1% (p<0.005) in Experimental group and 15.1±3.7% to 14.2±3.8% in the Placebo group (p<NS), as shown in FIG. 3. The effect of Xanthigen™ on liver fat content in obese subjects with nonalcoholic fatty liver disease (NAFLD) is seen in FIG. 3, where open triangles represent results obtained with patients on placebo and open squares represent patients receiving Xanthigen.

There was also a significant improvement in liver histology regarding features of NAFLD, steatosis, inflammation and fibrosis. Thus, these results strongly indicate that the Experimental Sample promotes significant liver fat reduction and normalize the level of plasma ALT, AST, GGT enzymes.

### Plasma C-Reactive Protein Assay

C-reactive protein (CRP) is a marker of acute inflammation and is generally used as a measure of inflammatory disease. Furthermore, the levels of plasma CRP increase in obesity and type 2 diabetes (Ford et al., 1999; Hak et al., 1999). In addition, results of recent studies also indicated that an inflammatory processes increased insulin resistance (Fiesta et al. 2000) and stimulated formation of visceral fat (Yudkin et al., 1999; Pradhan et al., 2001; Barzilay et al., 2001; Freeman et al., 2002). Thus, elevated levels of CRP predict the development of insulin-resistance, metabolic syndrome, type 2 diabetes, which supports a possible role for inflammation in diabetogenesis.

C-reactive protein was measured in aliquots of blood plasma collected and stored at 70° C. A high-sensitivity, two-site enzyme-linked immunoassay was developed with use of a peroxidase-conjugated rabbit antihuman C-reactive protein antibody (DK2600, Dako, Glostrup, Denmark) and a polyclonal anti-C-reactive protein capture antibody. The lower limit of the working range of the assay was 0.1 mg per liter as described by Macyii et al. (1997). CRP standard serum was used for calibration.

### Results

The effect of the Experimental Sample on plasma concentrations of pro-inflammatory C-reactive proteins (CRP) in the obese subjects with NAFLD is summarized in Table 5. This result indicates that the Experimental Sample supplementation significantly reduced plasma CRP from 6.6±2.7 mg/l to 3.64±2.8 mg/L (p<0.05) during 16 weeks of the trial, while in the placebo group from 6.3±2.7 mg/l to 5.44±2.1 mg/L (p<NS). This result strongly indicates that Experimental Sample possesses anti-inflammatory properties.

Effect of Experimental Sample on Blood Pressure in Obese Subjects with NAFLD

Several large epidemiological studies have documented the association between body weight and blood pressure (Stamler et al., 1989; Dyer & Elliott, 1989; Van Gaal et al., 1997). The supplementation of Experimental Sample reduced significantly both systolic and diastolic blood pressure in obese subjects, while no change in blood pressure was observed in the Placebo group (Table 5). Systolic Blood Pressure of obese subjects with NAFLD was reduced from 138±6 mm Hg to 119±6 mm Hg (p<0.05) during 16 weeks of Experimental Sample supplementation and Diastolic Blood Pressure was reduced from 91±4 mm Hg to 79±3 mm Hg (p<0.05).

On the other hand, no such positive changes in Systolic and Diastolic Blood Pressure was observed in the Placebo group. The correlation between reduction of the liver fat and normalization of blood pressure was largely anticipated because majority of obese subjects also develop hypertension.

TABLE 5
Pre-clinical and Post-clinical characteristics of obese subjects
with fatty liver

| | | Preclinical | Post- | Post- |
|---|---|---|---|---|
| | Preclinical | Experimental | clinical | clinical |
| | Placebo, | Sample, | Placebo, | Experimental |
| Variable | n = 36 | n = 36 | n = 36 | Sample, n = 36 |
| Age, yr | 37.4 ± 2.8 | 36.1 ± 2.1 | | |
| Mass, kg | 93.5 ± 2.4 | 94.1 ± 2.1 | 92.1 ± 2.8 | 87.2 ± 3.7 |
| Fat mass, kg | 42.1 ± 1.7 | 42.3 ± 2.2 | 41.2 ± 2.3 | 37.9 ± 2.9* |
| Liver Fat, % | 15.1 ± 3.7 | 15.3 ± 4.1 | 14.2 ± 3.8 | 9.4 ± 3.1* |
| CRP, mg/L | 6.3 ± 2.7 | 6.18 ± 2.4 | 5.44 ± 2.1 | 3.64 ± 2.8* |
| ALT units/L | 51 ± 9 | 48 ± 7 | 40 ± 6 | 26 ± 7* |
| AST units/L | 53 ± 7 | 51 ± 5 | 46 ± 6 | 29 ± 6* |
| GGT units/L | 49 ± 5 | 47 ± 7 | 46 ± 6 | 31 ± 5* |
| Systolic | 136 ± 5 | 138 ± 6 | 124 ± 4 | 119 ± 6* |
| Blood | | | | |
| Pressure mmHg | | | | |
| Diastolic | 88 ± 2 | 91 ± 4 | 85 ± 3 | 79 ± 3* |
| Blood | | | | |
| Pressure mmHg | | | | |

Values are means ± SE.
*P < 0.05

### Example 4

### Effect of Experimental Sample on Liver Fat and Plasma Serum Enzyme in Obese Subjects with Healthy Liver

Table 6 summarizes the effect of Experimental Sample on biochemical and physiological characteristics of the obese subjects with healthy liver fat levels, who participated in 16 weeks clinical trial. The selection criteria for obese subjects with healthy liver fat levels were the content of liver fat less than 5.3±1.5%. Thirty eight (n=38) obese pre-menopausal female subjects, with an average body weight 94.5±2.1 kgs, average age of 34±5.7 years, and liver fat content 5.3±1.5% were recruited to take part in a double-blind, placebo-controlled, randomized clinical trial.

### Results

The supplementation of 600 mg of Experimental Sample during 16 weeks of trial reduced liver fat content in obese subjects from 5.1±1.5% to 3.4±1.8% (p<0.05), and from 5.3±1.1% to 4.6±1.4% in the placebo group (p<NS), as shown in FIG. 3. The effect of Xanthigen™ on liver fat content in obese subjects with healthy livers (NLD) is seen in FIG. 3, where filled triangles represent results obtained with patients on placebo and filled squares represent patients receiving Xanthigen. Obese subjects with healthy liver fat levels also had slightly elevated levels of plasma AST, ALT and GGT, although the absolute values of these marker enzymes were significantly lower than those observed in the obese subjects with NAFLD (Table 6).

The Experimental Sample reduced both systolic and diastolic blood pressure in obese subjects with healthy liver as we observed previously in subjects with NAFLD (Table 6). Systolic blood pressure of obese subjects with healthy liver was reduced from 128±6 mm Hg to 112±6 mm Hg (p<0.05) during 16 weeks of Experimental Sample supplementation and Diastolic Blood Pressure was reduced from 93±2 mm Hg to 77±3 mm Hg (p<0.05). No significant change in blood pressure was observed in the Placebo group.

TABLE 6
Pre-clinical and Post-clinical characteristics of
obese subjects with healthy liver

| | | Preclinical | | Post-clinical |
|---|---|---|---|---|
| | Preclinical | Experimental | Post-clinical | Experimental |
| | Placebo | Sample | Placebo, | Sample |
| Variable | n = 19 | n = 19 | n = 19 | n = 19 |
| Age, years | 34.7 ± 3.5 | 35.7 ± 3.2 | | |
| Mass, kg | 93.9 ± 1.4 | 94.5 ± 2.1 | 92.5 ± 1.5 | 88.2 ± 1.9* |
| Fat mass, kg | 42.7 ± 2.4 | 43.3 ± 2.9 | 41.1 ± 2.9 | 38.1 ± 3.2* |
| Liver Fat (%) | 5.3 ± 1.1 | 5.1 ± 1.5 | 4.6 ± 1.4 | 3.4 ± 1.8* |
| ALT units/L | 31 ± 9 | 33 ± 7 | 28 ± 6 | 26 ± 7 |
| AST units/L | 33 ± 7 | 38 ± 5 | 29 ± 6 | 29 ± 2 |
| GGT units/L | 29 ± 3 | 27 ± 3 | 26 ± 2 | 21 ± 3 |
| Systolic | 126 ± 7 | 128 ± 6 | 128 ± 4 | 112 ± 6* |
| Blood Pressure | | | | |
| mmHg | | | | |
| Diastolic | 92 ± 4 | 93 ± 2 | 89 ± 4 | 77 ± 3* |
| Blood Pressure | | | | |
| mmHg | | | | |

Values are means ± SE.
*P < 0.05

1. A method of slowing the aging process in a mammalian subject, comprising:
activating at least one member of the sirtuin family of proteins, wherein said activating comprises:
administering to said subject an effective amount of a synergistic combination of fucoxanthin and punicic acid.

2. The method recited in claim 1, wherein said administering comprises:
administering an effective amount of a synergistic combination of fucoxanthin and punicic acid, wherein said fucoxanthin is administered as a component of an extract of a brown marine vegetable.

3. The method recited in claim 1, wherein said administering comprises:
administering an effective amount of a synergistic combination of fucoxanthin and punicic acid, wherein said punicic acid is administered as a component of a pomegranate seed oil.

4. The method recited in claim 1, wherein said activating comprises activating at least one protein selected from the group consisting of Sirt1 and Sirt3 proteins; wherein said activating places cells into a state of hibernation by deacetylating proteins.

5. The method recited in claim 1, wherein said activating comprises activating at least one mitochondrial protein selected from the group consisting of Sirt3, Sirt4, and Sirt5.

6. The method recited in claim 1, wherein said activating further comprises activating a mitochondrial UCP-1 protein.

7. The method recited in claim 1, wherein said method further comprises treatment of non-alcoholic fatty liver disease with said synergistic combination.

8. The method recited in claim 1, wherein said method further comprises treatment of fatty liver condition with said synergistic combination.

9. The method recited in claim 1, wherein said method further comprises increasing energy expenditure rate with said synergistic combination.

10. The method recited in claim 1, wherein said method further comprises:
increasing the volume of brown adipose tissue and decreasing the volume of white adipose tissue through treatment with said synergistic combination.

11. A method of slowing the aging process in a mammalian subject, comprising:
placing cells into a state of hibernation by deacetylating proteins, wherein said deacetylating proteins is achieved by administering to said subject an effective amount of a synergistic combination of fucoxanthin and punicic acid.

12. The method recited in claim 1, wherein the synergistic combination further comprises at least one omega-3 fatty acid.

13. The method recited in claim 1, wherein the synergistic combination further comprises at least one omega-3 fatty acid derived from brown algae.

14. The method recited in claim 1, wherein the synergistic combination comprises fucoxanthin derived from brown algae and at least one omega-3 fatty acid derived from brown algae.

15. The method recited in claim 1, wherein the synergistic combination comprises synthetically produced fucoxanthin and at least one omega-3 fatty acid derived from brown algae.

16. The method recited in claim 1, wherein the synergistic combination comprises:
fucoxanthin, alone or in combination with extracts of marine brown algae; and
punicic acid, alone or in combination with pomegranate seed oil.

17. The method recited in claim 1, wherein the synergistic combination comprises:
said synergistic combination of fucoxanthin and punicic acid;
wherein said fucoxanthin is included as a component of at least one extract of marine brown algae; and
wherein said punicic acid is included as a component of pomegranate seed oil.

18. A method as recited in claim 1, wherein said administering comprises topically applying or parenterally administering the synergistic combination to said subject.

19. A method as recited in claim 1, wherein said administering comprises orally administering the synergistic combination to said subject.

20. A method as recited in claim 1, wherein said subject is a human.

21. A method of improving body composition in a mammalian subject, comprising:
activating at least one member of the sirtuin family of proteins, wherein said activating comprises:
administering to said subject an effective amount of a synergistic combination of fucoxanthin and punicic acid.

22. A method of activating Sirt1 in adipose tissue, comprising:
contacting said tissue with an effective amount of a synergistic combination of fucoxanthin and punicic acid;
wherein said synergistic composition comprises:
50 wt. % of an extract of a brown marine vegetable, said extract comprising 30 wt. % of marine vegetable oil, and 0.5 to 5 wt. % fucoxanthin; and
50 wt. % of an pomegranate seed oil, said pomegranate seed oil comprising 60-80 wt. % of punicic acid.

23. A method as recited in claim 1, wherein said extract comprises 30 wt. % of marine vegetable oil, and 0.8 wt. % fucoxanthin.
